(19) United States
(12) Patent Application Publication
(10) Pub. No.: US 2005/0148781 A1
(43) Pub. Date: Jul. 7, 2005

(54) Process for the asymmetric synthesis of dihydrobenzoxathins

(76) Inventors: Zhiguo Jake Song, Edison, NJ (US); Marjorie See Waters, Cranbury, NJ (US)

Correspondence Address: MERCK AND CO., INC, P O BOX 2000, RAHWAY, NJ, 07065-0907, US

(21) Appl. No.: 10/967,773

(22) Filed: Oct. 18, 2004

Related U.S. Application Data
(60) Provisional application No. 60/516,441, filed on Oct. 31, 2003.

(51) Int. Cl. C07D 327/06

(52) U.S. Cl. 549/15

(57) ABSTRACT

This invention relates to an asymmetric synthesis of sulfides via a chiral sulfoxide directed stereospecific reduction of α,β-unsaturated sulfoxide to the saturated sulfide. These sulfides can be useful as selective estrogen receptor modulators or anti-neoplastic agents.

### BACKGROUND OF THE INVENTION

This invention relates to an asymmetric synthesis of sulfides via a chiral sulfoxide directed stereospecific reduction of α,β-unsaturated sulfoxide to the saturated sulfide. These sulfides can be useful as selective estrogen receptor modulators or anti-neoplastic agents.

### SUMMARY OF THE INVENTION

The present invention relates to processes for the synthesis of compounds of formula I:

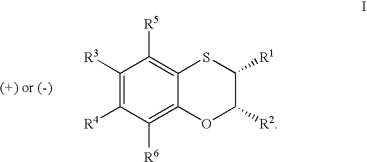

### DETAILED DESCRIPTION OF THE INVENTION

The present invention relates to processes for the synthesis of compounds of formula I: 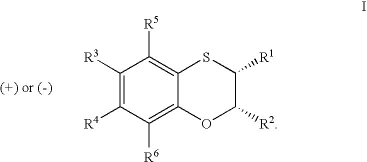
wherein R¹ and R² are selected from the group consisting of Hydrogen, C₁₋₅ alkyl, C₃₋₈ cycloalkyl, C₂₋₅ alkenyl, C₂₋₅ alkynyl, C₃₋₈ cycloalkenyl, phenyl, heteroaryl and heterocyclyl, wherein said alkyl, cycloalkyl, alkenyl, alkynyl, cycloalkenyl, phenyl, heteroaryl and heterocyclyl can be optionally substituted with C₁₋₅ alkyl, C₃₋₈ cycloalkyl, CF₃, phenyl, heteroaryl, heterocyclyl, —OR⁷, halogen, amino, C₁₋₅ alkylthio, thiocyanato, cyano, —CO₂H, —COOC₁₋₅ alkyl, —COC₁₋₅ alkyl, —CONZ₂, —SO₂NZ₂ or —SO₂C₁₋₅ alkyl;
R³ and R⁴, R5, R6 are each independently selected from the group consisting of hydrogen, C₁₋₅ alkyl, C₃₋₈ cycloalkyl, C₂₋₅ alkenyl, C₂₋₅ alkynyl, C₃₋₈ cycloalkenyl, phenyl, heteroaryl, heterocyclyl, CF₃, —OR⁷, halogen, C₁₋₅ alkylthio, thiocyanato, cyano, —CO₂H, —COOC₁₋₅ alkyl, —COC₁₋₅ alkyl, —CONZ₂, —SO₂NZ₂, and —SO₂C₁₋₅ alkyl, wherein said alkyl, alkenyl, alkynyl, cycloalkyl, cycloalkenyl, phenyl, heteroaryl and heterocyclyl can be optionally substituted with C₁₋₅ alkyl, C₃₋₈ cycloalkyl, CF₃, phenyl, heteroaryl, heterocyclyl, —OR⁷, halogen, amino, C₁₋₅ alkylthio, thiocyanato, cyano, —CO₂H, —COOC₁₋₅ alkyl, —COC₁₋₅ alkyl, —CONZ₂, —SO₂NZ₂ or —SO₂C₁₋₅ alkyl;
R⁷ is selected from the group consisting of hydrogen, C₁₋₅ alkyl, benzyl, methoxymethyl, triorganosilyl, C₁₋₅ alkylcarbonyl, alkoxycarbonyl and CONZ₂;
each Z is independently selected from the group consisting of hydrogen, C₁₋₅ alkyl, trifluoromethyl, wherein said alkyl can be optionally substituted with C₁₋₅ alkyl, CF₃, —OR⁷, halogen, amino, C₁₋₅ alkylthio, —COOC₁₋₅ alkyl, —COC₁₋₅ alkyl, —CONV₂, —SO₂NV₂ or —SO₂C₁₋₅ alkyl;
   both Zs and the nitrogen to which they are attached may be taken together to form a 3-8 membered ring, said ring may optionally contain atoms selected from the group consisting of carbon, oxygen, sulfur, and nitrogen, wherein said ring may either be saturated or unsaturated, and the carbon atoms of said ring maybe optionally substituted with one to three substituents selected from the group consisting of C₁₋₅ alkyl, CF₃, —OR⁷, halogen, amino, C₁₋₅ alkylthio, thiocyanato, cyano, —CO₂H, —COOC₁₋₅ alkyl, —COC₁₋₅ alkyl, —CONV₂, —SO₂NV₂, and —SO₂C₁₋₁₅ alkyl;
and each V is independently selected from the group consisting of C₁₋₅ alkyl, CF₃, —OR⁷, halogen, amino, C₁₋₅ alkylthio, —COOC₁₋₅ alkyl, —COC₁₋₅ alkyl, and —SO₂C₁₋₅ alkyl.

The first step in the synthesis of compounds of formula I comprises oxidizing a vinyl sulfide of formula II to yield a vinyl sulfoxide of formula III.

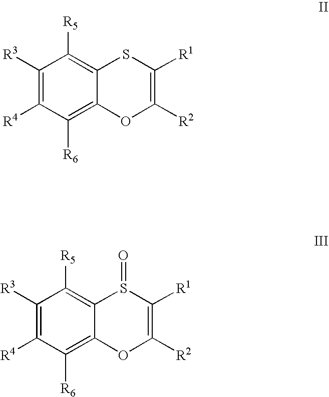

The oxidation is performed with a chiral ligand, an amine, water and a metal catalyst in a solvent, and an oxidizing agent.

Suitable chiral ligands for the oxidation include, but are not limited to, di(C₁₋₆ alkyl)tartrate, including diisopropyl tartrate and diethyl tartrate; 1,1′-bi-2-naphthol; hydrobenzoin; N,N′-dibenzyl tartramide; and α,α,α′,α′-tetraphenyl-1,3-dioxolane-4,5-dimethanol. In a class of the invention, the chiral ligand is diisopropyl tartrate or diethyl tartrate.

Suitable amines for the oxidation include, but are not limited to, trialkylamines, including diusopropylethylamine.

Suitable metal catalysts include, but are not limited to, titanium (IV) isopropoxide, titanium (IV) methoxide and titanium (IV) ethoxide. In a class of the invention, the metal catalyst is titanium (IV) isopropoxide.

Suitable solvents for the oxidation include, but are not limited to, THF, toluene, ethyl acetate, dichloromethane, isopropyl acetate, acetonitrile and chloroform. In a class of the invention, the solvent is THF.

Suitable oxidizing agents include, but are not limited to, cumene hydroperoxide and t-butyl hydroperoxide. In a class of the invention, the oxidizing agent is cumene hydroperoxide.

The oxidation can be performed between about 40 to about 50° C. In a class of the invention, the oxidation is performed between about 25 to about 30° C.

The next step comprises reducing the vinyl sulfoxide of formula III to yield the compounds of formula I.

The reduction is performed with a reducing agent. Suitable reducing agents include, but are not limited to, borane THF, borane dimethylsulfide and borane amine complexes, such as borane dimethylaniline.

The reduction can be performed between about 0 to about 20° C. In a class of the invention, the oxidation is performed at 10° C.

Definitions

As used herein, “alkyl” is intended to include both branched and straight-chain saturated aliphatic hydrocarbon groups having the specified number of carbon atoms. For example, C₁-C₁₀, as in “C₁-C₁₀ alkyl” is defined to include groups having 1, 2, 3, 4, 5, 6, 7, 8, 9 or 10 carbons in a linear or branched arrangement. For example, “C₁-C₁₀ alkyl” specifically includes methyl, ethyl, propyl, butyl, pentyl, hexyl, heptyl, octyl, nonyl, decyl, and so on. “Alkoxy” represents an alkyl group of indicated number of carbon atoms attached through an oxygen bridge.

The term “cycloalkyl” shall mean cyclic rings of alkanes of three to eight total carbon atoms, or any number within this range (i.e., cyclopropyl, cyclobutyl, cyclopentyl, cyclohexyl, cycloheptyl or cyclooctyl).

If no number of carbon atoms is specified, the term “alkenyl” refers to a non-aromatic hydrocarbon radical, straight or branched, containing from 2 to 10 carbon atoms and at least 1 carbon to carbon double bond. Preferably 1 carbon to carbon double bond is present, and up to 4 non-aromatic carbon-carbon double bonds may be present. Thus, “C₂-C₆ alkenyl” means an alkenyl radical having from 2 to 6 carbon atoms. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated.

The term “cycloalkenyl” shall mean cyclic rings of 3 to 10 carbon atoms and at least 1 carbon to carbon double bond (i.e., cyclopropenyl, cyclobutenyl, cyclopentenyl, cyclohexenyl, cycloheptenyl or cyclooctenyl).

The term “alkynyl” refers to a hydrocarbon radical straight or branched, containing from 2 to 10 carbon atoms and at least 1 carbon to carbon triple bond. Up to 3 carbon-carbon triple bonds may be present. Thus, “C₂-C₆ alkynyl” means an alkynyl radical having from 2 to 6 carbon atoms. Alkynyl groups include ethynyl, propynyl and butynyl. As described above with respect to alkyl, the straight or branched portion of the alkynyl group may contain triple bonds and may be substituted if a substituted alkynyl group is indicated.

In certain instances, substituents may be defined with a range of carbons that includes zero, such as (C₀-C₆)alkylaryl. If aryl is taken to be phenyl, this definition would include phenyl itself as well as —CH₂Ph, —CH₂CH₂Ph, CH(CH₃) CH₂CH(CH₃)Ph, and so on.

As used herein, “aryl” is intended to mean any stable monocyclic or bicyclic carbon ring of up to 10 atoms in each ring, wherein at least one ring is aromatic. Examples of such aryl elements include phenyl, naphthyl, tetrahydronaphthyl, indanyl, biphenyl, phenanthryl, anthryl or acenaphthyl. In cases where the aryl substituent is bicyclic and one ring is non-aromatic, it is understood that attachment is via the aromatic ring.

The term “heteroaryl”, as used herein, represents a stable monocyclic or bicyclic ring of up to 10 atoms in each ring, wherein at least one ring is aromatic and contains from 1 to 4 heteroatoms selected from the group consisting of O, N and S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

As appreciated by those of skill in the art, “halo” or “halogen” as used herein is intended to include chloro, fluoro, bromo and iodo.

The term “hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxyethyl, 2-hydroxypropyl, 3-hydroxypropyl, and the like.

The term “heterocyclyl” as used herein is intended to mean a 5- to 10-membered nonaromatic ring containing from 1 to 4 heteroatoms selected from the group consisting of O, N and S, and includes bicyclic groups. “Heterocyclyl” therefore includes, but is not limited to the following: imidazolyl, piperazinyl, piperidinyl, pyrrolidinyl, morpholinyl, thiomorpholinyl, tetrahydropyranyl, dihydropiperidinyl, tetrahydrothiophenyl and the like. If the heterocycle contains a nitrogen, it is understood that the corresponding N-oxides thereof are also emcompassed by this definition.

The present invention also includes N-oxide derivatives and protected derivatives of compounds of Formula I. For example, when compounds of Formula I contain an oxidizable nitrogen atom, the nitrogen atom can be converted to an N-oxide by methods well known in the art. Also when compounds of Formula I contain groups such as hydroxy, carboxy, thiol or any group containing a nitrogen atom(s), these groups can be protected with a suitable protecting groups. A comprehensive list of suitable protective groups can be found in T.W. Greene, Protective Groups in Organic Synthesis, John Wiley & Sons, Inc. 1981, the disclosure of which is incorporated herein by reference in its entirety. The protected derivatives of compounds of Formula I can be prepared by methods well known in the art.

The alkyl, alkenyl, alkynyl, cycloalkyl, aryl, heteroaryl and heterocyclyl substituents may be unsubstituted or unsubstituted, unless specifically defined otherwise. For example, a (C₁-C₆)alkyl may be substituted with one or more substituents selected from OH, oxo, halogen, alkoxy, dialkylamino, or heterocyclyl, such as morpholinyl, piperidinyl, and so on. In the case of a disubstituted alkyl, for instance, wherein the substituents are oxo and OH, the following are included in the definition: —(C═O)CH₂CH(OH)CH₃, —(C═O)OH, —CH₂(OH)CH₂CH(O), and so on.

In the compounds of the present invention, alkyl, alkenyl, alkynyl, cycloalkyl, cycloalkenyl, heterocyclyl and heteroaryl groups can be further substituted by replacing one or more hydrogen atoms be alternative non-hydrogen groups. These include, but are not limited to, halo, hydroxy, mercapto, amino, carboxy, cyano and carbamoyl.

The term “oxy” means an oxygen (O) atom. The term “thio” means a sulfur (S) atom. The term “oxo” means ═O. The term “oximino” means the ═N—O group. The term “keto” means carbonyl (C═O). The term “thiocyanato” refers to —SCN.

The term “substituted” shall be deemed to include multiple degrees of substitution by a named substitutent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

When any variable (e.g. V, Z etc.) occurs more than one time in any constituent, its definition on each occurrence is independent at every other occurrence. Also, combinations of substituents and variables are permissible only if such combinations result in stable compounds. Lines drawn into the ring systems from substituents indicate that the indicated bond may be attached to any of the substitutable ring carbon atoms. If the ring system is polycyclic, it is intended that the bond be attached to any of the suitable carbon atoms on the proximal ring only.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted with one or more substituents” should be taken to be equivalent to the phrase “optionally substituted with at least one substituent” and in such cases the preferred embodiment will have from zero to three substituents.

Under standard nonmenclature used throughout this disclosure, the terminal portion of the designated side chain is described first, followed by the adjacent functionality toward the point of attachment. For example, a C₁₋₅ alkylcarbonylamino C₁₋₆ alkyl substituent is equivalent to

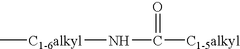

In choosing compounds of the present invention, one of ordinary skill in the art will recognize that the various substituents, i.e. R¹, R² and R³ are to be chosen in conformity with well-known principles of chemical structure connectivity.

The following generic scheme and specific examples are not intended to limit the present invention, but to illustrate aspects of the present invention. Those skilled in the art will readily understand that known variations of the conditions and processes of the following preparative procedures can be used to prepare these compounds. All temperatures are degrees Celsius unless otherwise noted. 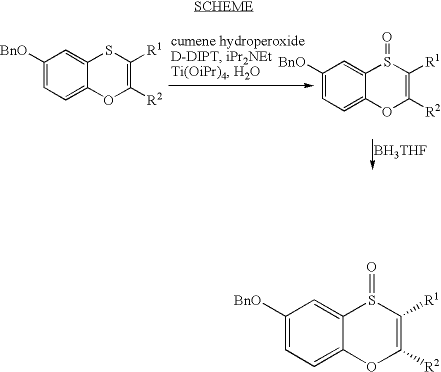
General Procedure for the Oxidation Reduction Sequence:

A stock solution was prepared and used multiple times for the different substrates: Catalyst stock solution: D-Diisopropyl tartrate (1.19 g, 5.06 mmol) and diisopropylethylamine (0.65 g, 5.0 mmol) were dissolved in 25 mL toluene. The solution was degassed and titanium isopropoxide (0.71 g, 2.5 mmol) was added subsurface. Water (0.045 mL, 2.5 mmol) was added to the mixture subsurface over 2 minutes.

Oxidation reduction reaction: The vinyl sulfide (2 mmol) was dissolved in 2 mL of the catalyst stock solution. Cumene hydroperoxide (87%, 370 uL, 2.6 mmol) was added and the reaction aged overnight until complete by HPLC. The reaction was cooled to 5-10° C. and 6.0 mL of 1 M BH₃.THF added dropwise. The solution was allowed to warm to room temperature. The reaction mixture was washed twice with 10 mL of 10% tartaric acid solution. The organic layer was concentrated in vacuo and purified either by column chromatography or crystallization.

### EXAMPLE 1

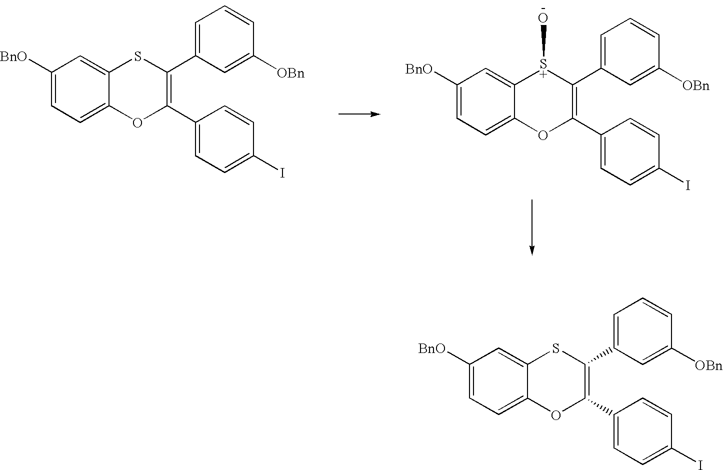

Vinyl sulfoxide To a 100-L round bottom flask was charged 60 L of THF (KF=64.5 μg/ml), D-diisopropyl tartrate (0.66 kg, 2.8 mol) and diisopropylethylamine (0.36 kg, 2.8 mol), 21.4 ml of water were added to the solution so the total amount of water present was 25.3 g (1.41 mol). The flask was degassed by two vacuum/nitrogen fill cycles. With vigorous stirring, titanium isopropoxide (0.40 kg, 1.41 mol) was transferred subsurface via reduced pressure from a 500 ml round bottom flask. The solution was aged at ambient overnight. Vinyl sulfide (6.00 kg, 9.37 mol) was added and the resulting yellow solution was warmed to 25° C. About 10% of the cumene hydroperoxide (CHP, 87%, 2.01 kg, 11.5 mol) was added over 5 minutes. After aging for 10 minutes, 60 g of seed was added. The mixture was aged for 15 minutes to ensure seedbed formation. The remaining CHP, from the 2.01 kg total, was added via addition funnel over 1 h. The slurry was held at 24-27° C. for 1 h, then allowed to cool to ambient and aged overnight. HPLC assay of the slurry shows 95.0 A % sulfoxide, 1.1 A % sulfone, and 3.9 A % sulfide starting material. The chiral assay indicated the sulfoxide has a 92.5% ee. The solid was isolated by filtration and the cake washed with 8 L of THF then 10 L of toluene. The solid was dried on the filter with nitrogen flow overnight, giving the title compound as an yellow solid (5.28 kg, 86% yield). HPLC indicated 95.4% pure, 99.8% ee. HPLC conditions (area %): column: PE 3×3 C18CR. mobile phase: A: 0.1% H₃PO₄. B: MeCN. Gradient: 45:55 A/B to 25:75 A/B over 8 min. Flow: 2.0 mL/min. Temp: 25° C. UV: 210 nm. Retention times: 3.5 min (vinyl sulfoxide), 4.4 min (vinyl sulfone), 7.3 min (vinyl sulfide). HPLC conditions (Chiral): Column: (R,R)-Whelk-O, 4.6×50 mm. Mobile Phase: 30% IPA in hexanes, isocratic. Flow 2.0 mL/min. Temp 35° C. UV: 210 nm. Retention times: 1.4 min (major), 3.35 min (minor). Mp 218-22° C. ¹H NMR (400 MHz, CDCl₃) δ 7.6-7.7 (m, 2H), 7.15-7.50 (m, 16H), 7.1(m, 1H), 6.9-7.0 (m, 2H), 5.16 (ABq, J=11.6 Hz, Δυ=19.9 Hz, 2H), 5.00 (ABq, J=11.7 Hz, Δυ=7.4 Hz, 2H). ¹³C NMR (101 MHz, CDCl₃) δ 159.10, 156.2, 151.7, 143.7, 137.5, 136.7, 136.4, 136.1, 132.8, 131.5, 130.1, 128.8, 128.6, 128.4, 128.1, 127.6, 127.5, 123.3, 122.3, 119.9, 116.6, 115.6, 113.2, 97.0, 71.0, 70.1. LC-MS (M+1)+/e 657, (M+23)/e 679. [α]²² _{Na}=−80 0 (0.10% CHCl₃). HRMS calcd for C₃₄H₂₆IO₄S 657.0596 found 657.0598.

Iododihydrobenzoxathiin To a 100-L cylindrical vessel equipped with internal cooling coil was charged 50 L of toluene and vinyl sulfoxide (5.00 kg, 7.62 mol). The slurry was cooled to 5° C., with cooling coils set at −20° C., BH₃.THF (1 M, 9.2 mol) added over 40 min. After aging 30 min at 5° C., the batch was gradually warmed to 10° C. The reaction was aged until complete dissolution, indicating completion of reaction. HPLC indicated >99.5% conversion. The resulting solution was warmed to 20° C. and quenched by addition of 20 L of 2 N HCl and agitated for 10 minutes (Caution: During the quench, evolution of presumably hydrogen gas and a small exotherm were noted). The aqueous layer was cut and the organic layer washed with 20 L 2 N HCl then 20 L water. The organic layer was filtered through a 5 micron inline filter and concentrated in vacuum in an 100 L round bottom flask equipped with distillation condenser at 40-50° C., 28-29 mmHg. The final batch volume was ˜15 L and the KF was 204 μg/mL. At 40° C., 3.7 L of n-heptane was added and the solution was allowed to cool to 25° C. and 5 g of seed added. The resulting mixture was aged overnight to ensure seedbed formation. The remaining 26.2 L of n-heptane was added by addition funnel over 1.5 h at 20° C. After aging for 1 h, the solid was isolated by filtration and the cake washed with 8 L of 4:1 n-heptane/toluene then 2×8 L of n-heptane. The solid was dried on the pressure filter under nitrogen, giving the title compound as an off-white solid (4.31 kg, 88% yield). HPLC indicated 99.8% pure, 99.9% ee. HPLC conditions (Area %) same as last step. Retention times: 3.5 min (vinyl sulfoxide), 6.0 min (reduced sulfide). HPLC conditions (Chiral): Column: Chiralcel OD-H, 4.6×250 mm. Mobile Phase: 5% EtOH/0.5% IPA/94.5% hexanes, isocratic. Flow 2.0 mL/min. Temp 20° C. UV: 210 nm. Retention times: 5.5 min (minor), 6.8 min (major). Mp 99-100° C., ¹H NMR (400 MHz, CDCl₃) δ 7.3-7.6 (m, 11H), 7.04 (t, J=8.0 Hz, 1H), 6.92 (d, 8.9 Hz, 1H), 6.70-6.85 (m, 5H), 6.5-6.6 (m, 2H), 5.43(d, J=2.2 Hz, 1H), 5.04 (s, 2H), 4.85 (ABq, J=11.9 Hz, Δυ=1.2 Hz, 2H), 4.33 (d, J=2.3 Hz, 1H). ¹³C NMR (101 MHz, CDCl₃) δ 158.3, 154.0, 146.2, 139.3, 138.4, 137.1, 136.9, 128.9, 128.7, 128.6, 128.4, 128.0, 127.6, 127.5, 121.8, 119.5, 119.1, 115.5, 114.7, 113.1, 112.0, 93.6, 78.7, 70.7, 69.9, 47.9. [α]²² _{Na}=+236 0 (0.10% MeCN). HRMS calcd for C₃₄H₂₈IO₃S 643.0804 found 643.0807.

### EXAMPLE 2

Using the procedures described in the generic scheme and example above, and knowledge available to those skilled in the art, the following additional compounds can be prepared:

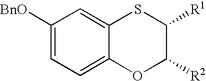

| R₁ | R₂ |
|---|---|
| H | Me |
| H | tBu |
| H | Ph |
| H | 4-MePh |
| H | 4-BrPh |
| H | 4-MeOPh |
| Me | Me |
| Me | Ph |
| Ph | Ph |
| Ph | 4-BrPh |
| 4-MeOPh | 4-MeOPh |
| Ph | H |

1. A process for making compounds of formula I: 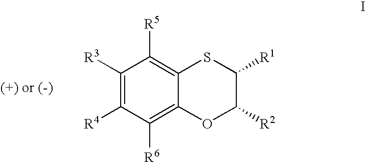
comprising the steps of:
a. oxidizing a vinyl sulfide of formula II to yield a vinyl sulfoxide of formula III; and 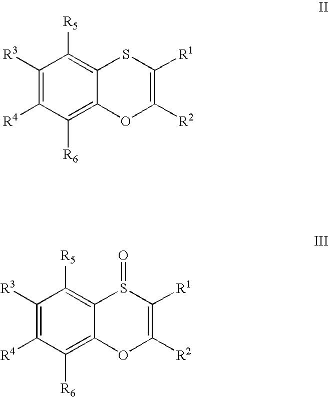
b. reducing the vinyl sulfoxide of formula III to yield the compounds of formula I;
wherein R¹ and R² are selected from the group consisting of Hydrogen, C₁₋₅ alkyl, C₃₋₈ cycloalkyl, C₂₋₅ alkenyl, C₂₋₅ alkynyl, C₃₋₈ cycloalkenyl, phenyl, heteroaryl and heterocyclyl, wherein said alkyl, cycloalkyl, alkenyl, alkynyl, cycloalkenyl, phenyl, heteroaryl and heterocyclyl can be optionally substituted with C₁₋₅ alkyl, C₃₋₈ cycloalkyl, CF₃, phenyl, heteroaryl, heterocyclyl, —OR⁷, halogen, amino, C₁₋₅ alkylthio, thiocyanato, cyano, —CO₂H, —COOC₁₋₅ alkyl, —COC₁₋₅ alkyl, —CONZ₂, —SO₂NZ₂ or —SO₂C₁₋₅ alkyl;
R³ and R⁴, R5, R6 are each independently selected from the group consisting of hydrogen, C₁₋₅ alkyl, C₃₋₈ cycloalkyl, C₂₋₅ alkenyl, C₂₋₅ alkynyl, C₃₋₈ cycloalkenyl, phenyl, heteroaryl, heterocyclyl, CF₃, —OR⁷, halogen, C₁₋₅ alkylthio, thiocyanato, cyano, —CO₂H, —COOC₁₋₅ alkyl, —COC₁₋₅ alkyl, —CONZ₂, —SO₂NZ₂, and —SO₂C₁₋₅ alkyl, wherein said alkyl, alkenyl, alkynyl, cycloalkyl, cycloalkenyl, phenyl, heteroaryl and heterocyclyl can be optionally substituted with C₁₋₅ alkyl, C₃₋₈ cycloalkyl, CF₃, phenyl, heteroaryl, heterocyclyl, —OR⁷, halogen, amino, C₁₋₅ alkylthio, thiocyanato, cyano, —CO₂H, —COOC₁₋₅ alkyl, —COC₁₋₅ alkyl, —CONZ₂, —SO₂NZ₂ or —SO₂C₁₋₅ alkyl;
R⁷ is selected from the group consisting of hydrogen, C₁₋₅ alkyl, benzyl, methoxymethyl, triorganosilyl, C₁₋₅ alkylcarbonyl, alkoxycarbonyl and CONZ₂;
each Z is independently selected from the group consisting of hydrogen, C₁₋₅ alkyl, trifluoromethyl, wherein said alkyl can be optionally substituted with C₁₋₅ alkyl, CF₃, —OR⁷, halogen, amino, C₁₋₅ alkylthio, —COOC₁₋₅ alkyl, —COC₁₋₅ alkyl, —CONV₂, —SO₂NV₂ or —SO₂C₁₋₅ alkyl;
both Zs and the nitrogen to which they are attached may be taken together to form a 3-8 membered ring, said ring may optionally contain atoms selected from the group consisting of carbon, oxygen, sulfur, and nitrogen, wherein said ring may either be saturated or unsaturated, and the carbon atoms of said ring maybe optionally substituted with one to three substituents selected from the group consisting of C₁₋₅ alkyl, CF₃, —OR⁷, halogen, amino, C₁₋₅ alkylthio, thiocyanato, cyano, —CO₂H, —COOC₁₋₅ alkyl, —COC₁₋₅ alkyl, —CONV₂, —SO₂NV₂, and —SO₂C₁₋₅ alkyl;
and each V is independently selected from the group consisting of C₁₋₅ alkyl, CF₃, —OR⁷, halogen, amino, C₁₋₅ alkylthio, —COOC₁₋₅ alkyl, —COC₁₋₅ alkyl, and —SO₂C₁₋₅ alkyl.

2. The process of claim 1 wherein the oxidation is performed with a chiral ligand, an amine, water and a metal catalyst in a solvent, and an oxidizing agent.

3. The process of claim 2 wherein the chiral ligand is selected from di(C₁₋₁₆ alkyl)tartrate, 1,1′-bi-2-naphthol, hydrobenzoin, N,N′-dibenzyl tartramide, and α,α,α′,α′-tetraphenyl-1,3-dioxolane-4,5-dimethanol.

4. The process of claim 3 wherein the chiral ligand is diisopropyl tartrate or diethyl tartrate.

5. The process of claim 2 wherein the amine is a trialkylamine

6. The process of claim 5 wherein the amine is diisopropylethylamine.

7. The process of claim 2 wherein the metal catalyst is selected from titanium (IV) isopropoxide, titanium (IV) methoxide or titanium (IV) ethoxide.

8. The process of claim 7 wherein the metal catalyst is titanium (IV) isopropoxide.

9. The process of claim 2 wherein the solvent is selected from THF, toluene, ethyl acetate, dichloromethane, isopropyl acetate, acteonitrile, or chloroform.

10. The process of claim 9 wherein the solvent is THF.

11. The process of claim 2 wherein the oxidizing agent is cumene hydroperoxide or t-butyl hydroperoxide.

12. The process of claim 11 wherein the oxidizing agent is cumene hydroperoxide.

13. The process of claim 2 wherein the oxidation is performed between about −40° C. and about 50° C.

14. The process of claim 13 wherein the oxidation is performed between about 25° C. and about 30° C.

15. The process of claim 1 wherein the reduction is performed with a reducing agent.

16. The process of claim 15 wherein the reducing agent is selected from borane THF, borane dimetylsulfide or borane dimethylaniline

17. The process of claim 16 wherein the reducing agent is borane THF.

18. The process of claim 15 wherein the reduction is performed between about 0° C. to about 20° C.

19. The process of claim 18 wherein the reduction is performed at about 10° C.
